(19) United States
(12) Patent Application Publication
(10) Pub. No.: US 2013/0005900 A1
(43) Pub. Date: Jan. 3, 2013

(54) GELS AND SOFT ELASTOMERS MADE WITH NATURAL OIL BASED POLYOLS

(75) Inventors: Gerhard Mueller, Adlington (GB); Verena M.T. Thiede, Muenster (DE)

(73) Assignee: DOW GLOBAL TECHNOLOGIES LLC, Midland, MI (US)

(21) Appl. No.: 13/634,483

(22) PCT Filed: Mar. 10, 2011
(86) PCT No.: PCT/US11/27928

§ 371 Date: Sep. 12, 2012

Related U.S. Application Data
(60) Provisional application No. 61/313,290, filed on Mar. 12, 2010.

(51) Int. Cl. C08G 18/06; C07C 31/20; C07C 59/44; C07C 69/533; C07C 59/42; C08L 75/04; C07C 35/14

(52) U.S. Cl. 524/590; 528/85; 528/83; 568/852; 568/833; 560/224; 562/587

(57) ABSTRACT

Embodiments of the invention provide for polyurethane gels or soft elastomers which incorporate renewable resources without compromising the physical and chemical properties of the gels or soft elastomers, such as resistance to many chemicals.

### CROSS-REFERENCE TO RELATED APPLICATIONS

This application claims benefit of U.S. Provisional Patent Application Ser. No. 61/313,290, filed Mar. 12, 2010, entitled “GELS AND SOFT ELASTOMERS MADE WITH NATURAL OIL BASED POLYOL” which is herein incorporated by reference.

### FIELD OF THE INVENTION

Embodiments of the invention relate to polyurethane products, more specifically to gels or soft elastomers made using natural oil based polyols.

### BACKGROUND OF THE INVENTION

Polyurethane gels or soft elastomers are used in cushioning, mattresses, dampening inserts fore shoes, mouse pads, wrist pads and the like. Polyether polyols based on the polymerization of alkylene oxides, polyester polyols, or combinations thereof, are together with isocyanates the major components of such polyurethane systems. Most commercially available polyols are produced from petroleum. However, the depletion of petroleum combined with its increasing price in our modern societies has encouraged researchers and governments to explore new ways to produce today's polymeric materials from renewable natural resources. Furthermore, the polyurethane systems using only petroleum based polyols may have limited chemical resistant properties. Therefore, there is a need for a method of producing polyurethane gels or soft elastomers that result in an increased amount of renewable resources in the final product while maintaining the quality of the products.

### SUMMARY OF THE INVENTION

Embodiments of the invention provide for polyurethane gels or soft elastomers which incorporate renewable resources without compromising the physical and chemical properties of the gels or soft elastomers, such as resistance to many chemicals.

In one embodiment, a polyurethane product including a reaction product of at least one first polyol composition and at least one prepolymer composition is provided. The prepolymer composition may include the reaction product of at least a second polyol composition and at least one isocyanate composition. At least one of the first polyol composition and the second polyol composition may include at least one polyol derived from a natural oil. The polyurethane product has a Shore 00 hardness as measured according to ASTM D 2240 of between about 0 Shore 00 and about 80 Shore 00 and is one of a gel and a soft eleastomer.

In one embodiment, a method of forming a polyurethane product is provided. The method includes providing at least a first polyol composition, forming at least one prepolymer composition by combing at least a second polyol composition with at least one isocyanate composition, and reacting the at least first polyol composition with the at least one prepolymer composition. At least one of the first polyol composition and the second polyol composition includes at least one polyol derived from a natural oil. The polyurethane product has a Shore 00 hardness as measured according to ASTM D 2240 of between about 0 Shore 00 and about 80 Shore 00 and is one of a gel and a soft eleastomer.

In certain embodiments, the at least one polyol derived from a natural oil includes at least one of a hydroxymethylated fatty acid and a hydroxymethylated fatty acid ester.

In certain embodiments, the at least one polyol derived from a natural oil includes the reaction product of at least one of a hydroxymethylated fatty acid and a hydroxymethylated fatty acid ester and an initiator compound having a OH functionality, primary amine functionality, secondary amine functionality, or combination OH, primary, or secondary amine functionality, of between about 2 and about 4. The initiator compound may be selected from ethylene glycol, 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, glycerine, trimethylol propane, 1,2,6-hexane triol, 1,2,4-butane triol, trimethylolethane, pentaerythritol, quinitol, mannitol, sorbitol, methyl glycoside, diethylene glycol, triethylene glycol, tetraethylene glycol, dipropylene glycol, dibutylene glycol and combinations thereof. In one embodiment the initiator compound is a mixture of 1,3-cyclohexane dimethanol and 1,4-cyclohexane dimethanol.

In certain embodiments, the at least one polyol derived from a natural oil comprises at least an aliphatic polyester polyol prepared by the condensation of at least one diol with adipic, glutaric, succinic, dimer acid, or combination thereof. The at least one diol may be selected from 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, and combinations thereof.

In certain embodiments, the at least one first polyol composition comprises the at least one polyol derived from a natural oil, and/or the at least one second polyol composition comprises the at least one polyol derived from a natural oil. In certain embodiments, the at least one second polyol composition comprises at least one polyol derived from a natural oil, and which is different from the at least one polyol derived from a natural oil of the first polyol composition.

In certain embodiments, at least one of the first polyol composition and the second polyol composition further includes a polyether polyol having a nominal functionality of 2 to 8.

In one embodiment, the polyurethane product as described above has a Shore 00 hardness of less than about 50 Shore 00.

In one embodiment, the polyurethane product as described above further includes at least one foil adhered to the at least one of a gel and a soft elastomer.

### DESCRIPTION OF EMBODIMENTS OF THE INVENTION

Embodiments of the invention provide for polyurethane gels or soft elastomers made using natural oil based polyols and/or natural acid based polyols. The gels or elastomers are a so-called two component elastomers, as they are made from reacting at least a first polyol composition with at least one prepolymer composition. The prepolymer composition may have at least one urethane group, and may be the reaction product of at least one isocyanate and at least a second polyol composition. The first polyol composition and the second polyol composition may be the same or different, with at least one, or both, of the first or second polyol compositions including at least one natural oil based polyol (NOBP).

Natural oil based polyols (NOBP) are polyols based on or derived from renewable feedstock resources such as natural plant vegetable seed oils. The renewable feedstock resources may also include genetically modified (GMO) plant vegetable seed oils and/or animal source fats. Such oils and/or fats are generally comprised of triglycerides, that is, fatty acids linked together with glycerol. Preferred are vegetable oils that have at least about 70 percent unsaturated fatty acids in the triglyceride. Preferably the natural product contains at least about 85 percent by weight unsaturated fatty acids. Examples of preferred vegetable oils include, for example, those from castor, soybean, olive, peanut, rapeseed, corn, sesame, cotton, canola, safflower, linseed, palm, grapeseed, black caraway, pumpkin kernel, borage seed, wood germ, apricot kernel, pistachio, almond, macadamia nut, avocado, sea buckthorn, hemp, hazelnut, evening primrose, wild rose, thistle, walnut, sunflower, jatropha seed oils, or a combination thereof. Additionally, oils obtained from organisms such as algae may also be used. A combination of vegetable, algae, and animal based oils/fats may also be used.

For use in the production of polyurethane products, the natural material may be modified to give the material isocyanate reactive groups or to increase the number of isocyanate reactive groups on the material. Preferably such reactive groups are a hydroxyl group.

The modified natural oil derived polyols may be obtained by a multi-step process wherein the animal or vegetable oils/fats are subjected to transesterification and the constituent fatty acids recovered. This step is followed by hydroformylating carbon-carbon double bonds in the constituent fatty acids followed by reduction to form hydroxymethyl groups. Suitable hydroformylation/reduction methods are described in U.S. Pat. Nos. 4,731,486, 4,633,021, and 7,615,658, for example. The hydroxymethylated fatty acids or esters thereof are herein labeled “monomers” which form one of the building blocks for the natural oil based polyol. The monomers may be a single kind of hydroxymethylated fatty acid and/or hydroxymethylated fatty acid methyl ester, such as hydroxymethylated oleic acid or methylester thereof, hydroxymethylated linoleic acid or methylester thereof, hydroxymethylated linolenic acid or methylester thereof, α- and γ-linolenic acid or methyl ester thereof, myristoleic acid or methyl ester thereof, palmitoleic acid or methyl ester thereof, oleic acid or methyl ester thereof, vaccenic acid or methyl ester thereof, petroselinic acid or methyl ester thereof, gadoleic acid or methyl ester thereof, erucic acid or methyl ester thereof, nervonic acid or methyl ester thereof, stearidonic acid or methyl ester thereof, arachidonic acid or methyl ester thereof, timnodonic acid or methyl ester thereof, clupanodonic acid or methyl ester thereof, cervonic acid or methyl ester thereof, or hydroxymethylated ricinoleic acid or methylester thereof. In one embodiment, the monomer is hydroformulated methyloelate. Alternatively, the monomer may be the product of hydroformulating the mixture of fatty acids recovered from transesterifaction process of the animal or vegetable oils/fats to form hydroxymethylated fatty acids or methyl esters thereof. In one embodiment the monomer is hydroxymethylated soy bean fatty acids or methyl esters thereof which may have an average OH functionality of between about 0.9 and about 1.1 per fatty acid, preferably, the functionality is about 1. In another embodiment the monomer is hydroformulated castor bean fatty acids. In another embodiment, the monomer may be a mixture of selected hydroxymethylated fatty acids or methylesters thereof.

Alternatively, in other embodiments, at least one NOBP may be the polyol obtained by reacting the hydroxymethylated monomer with an appropriate initiator compound to form a polyester or polyether/polyester polyol. Such a multi-step process is commonly known in the art, and is described, for example, in PCT publication Nos. WO 2004/096882 and 2004/096883. The multi-step process results in the production of a polyol with both hydrophobic and hydrophilic moieties, which results in enhanced miscibility with both water and conventional petroleum-based polyols.

The initiator for use in the multi-step process for the production of the natural oil derived polyols may be any initiator used in the production of conventional petroleum-based polyols. Preferably the initiator is selected from the group consisting of neopentylglycol; 1,2-propylene glycol; trimethylolpropane; pentaerythritol; sorbitol; sucrose; glycerol; aminoalcohols such as ethanolamine, diethanolamine, and triethanolamine; alkanediols such as 1,6-hexanediol, 1,4-butanediol; 1,4-cyclohexane diol; 1,3-cyclohexanedimethanol, 1,4-cyclohexanedimethanol, 2,5-hexanediol; ethylene glycol; diethylene glycol, triethylene glycol; bis-3-aminopropyl methylamine; ethylene diamine; diethylene triamine; 9(1)-hydroxymethyloctadecanol, 1,4-bishydroxymethylcyclohexane; 8,8-bis(hydroxymethyl)tricyclo[5,2,1,0^{2,6}]decene; Dimerol alcohol (36 carbon diol available from Henkel Corporation); hydrogenated bisphenol; 9,9(10,10)-bishydroxymethyloctadecanol; 1,2,6-hexanetriol and combination thereof. Preferably the initiator is selected from the group consisting of glycerol; ethylene glycol; 1,2-propylene glycol; trimethylolpropane; ethylene diamine; pentaerythritol; diethylene triamine; sorbitol; sucrose; or any of the aforementioned where at least one of the alcohol or amine groups present therein has been reacted with ethylene oxide, propylene oxide or mixture thereof; and combination thereof. Preferably, the initiator is glycerol, trimethylopropane, pentaerythritol, sucrose, sorbitol, and/or mixture thereof. In one embodiment, the initiator is a mixture of 1,3-cyclohexanedimethanol and 1,4-cyclohexanedimethanol and is commercially available under the trade name UNOXOL from The Dow Chemical Company which is an approximate 1:1 mixture of (cis, trans) 1,3-cyclohexanedimethanol and (cis, trans) 1,4-cyclohexanedimethanol.

Other initiators include other linear and cyclic compounds containing an amine. Exemplary polyamine initiators include ethylene diamine, neopentyldiamine, 1,6-diaminohexane; bisaminomethyltricyclodecane; bisaminocyclohexane; diethylene triamine; bis-3-aminopropyl methylamine; triethylene tetramine various isomers of toluene diamine; diphenylmethane diamine; N-methyl-1,2-ethanediamine, N-Methyl-1,3-propanediamine, N,N-dimethyl-1,3-diaminopropane, N,N-dimethylethanolamine, 3,3′-diamino-N-methyldipropylamine, N,N-dimethyldipropylenetriamine, aminopropyl-imidazole.

In one embodiment, the initiators are alkoxlyated with ethylene oxide, propylene oxide, or a mixture of ethylene and at least one other alkylene oxide to give an alkoxylated initiator with a molecular weight between about 200 and about 6000, preferably between about 500 and about 5000. In one embodiment the initiator has a molecular weight of about 550, in another embodiment the molecular weight is about 625, and in yet another embodiment the initiator has a molecular weight of about 4600.

In one embodiment, at least one initiator is a polyether initiator having an equivalent weight of at least about 400 or an average at least about 9.5 ether groups per active hydrogen group, such initiators are described in copending Patent Application No. PCT/US09/37751, filed on Mar. 20, 2009, entitled “Polyether Natural Oil Polyols and Polymers Thereof” the entire contents of which are incorporated herein by reference.

The ether groups of the polyether initiator may be in poly(alkylene oxide) chains, such as in poly(propylene oxide) or poly(ethylene oxide) or a combination thereof. In one embodiment, the ether groups may be in a diblock structure of poly(propylene oxide) capped with poly(ethylene oxide).

In one embodiment, a NOBP is made with an initiator or combination of initiators having an average equivalent weight of between about 400 and about 3000 per active hydrogen group. All individual values and subranges between about 400 and about 3000 per active hydrogen group are included herein and disclosed herein; for example, the average equivalent weight can be from a lower limit of about 400, 450, 480, 500, 550, 600, 650, 700, 800, 900, 1000, 1200, or 1300 to an upper limit of about 1500, 1750, 2000, 2250, 2500, 2750, or 3000 per active hydrogen group.

Thus, in this embodiment, at least two of the natural oil based monomers are separated by a molecular structure having an average molecular weight of between about 1250 Daltons and about 6000 Daltons. All individual values and subranges between about 1250 Daltons and about 6000 Daltons are included herein and disclosed herein; for example, the average molecular weight can be from a lower limit of about 1250, 1500, 1750, 2000, 2250, 2500, 2750, 3000, or Daltons to an upper limit of about 3000, 3500, 4000, 4500, 5000, 5500, or 6000 Daltons.

To form the polyether initiator, the active hydrogen groups may be reacted with at least one alkylene oxide, such ethylene oxide or propylene oxide or a combination thereof; or a block of propylene oxide followed by a block of ethylene oxide, to form a polyether polyol by means within the skill in the art. The polyether initiator may be used as an initiator for reaction with at least one natural oil based monomer. Alternatively the initiator is reacted by means within the skill in the art to convert one or more hydroxyl groups to alternative active hydrogen groups, such as is propylene oxide.

Thus, in an embodiment, the natural oil based polyol may comprise at least two natural oil moieties separated by a molecular structure having at least about 19 ether groups or having an equivalent weight of at least about 400, preferably both. When the polyether initiator has more than 2 active hydrogen groups reactive with the natural oil or derivative thereof, each natural oil moiety is separated from another by an average of at least about 19 ether groups or a structure of molecular weight of at least about 400, preferably both.

The functionality of the resulting natural oil based polyols is above about 1.5 and generally not higher than about 6. In one embodiment, the functionality is below about 4. The hydroxyl number of the of the natural oil based polyols may be below about 300 mg KOH/g, preferably between about 50 and about 300, preferably between about 60 and about 200. In one embodiment, the hydroxyl number is below about 100.

The natural oil based polyols may alternatively be a polyester polyol produced from the condensation reaction of dimer fatty acids and non-dimer polycarboxilic acids with a polyhydroxy compound. Dimer fatty acids are known in the art, see for example, publication US 2005/0124711, the disclosure of which is incorporated herein by reference, and in general are dimerization products of mono- or polyunsaturated fatty acids and/or esters thereof. Such dimer fatty acids are dimers of C₁₀ to C₃₀, more preferably C₁₂ to C₂₄, and more preferably C₁₄ to C₂₂ alkyl chains. Suitable dimer fatty acids for producing the polyesters of the present invention include dimerization products of oleic acid, linoleic acid, linolenic acid, palmitoleic acid and elaidic acid. The dimerization products of the unsaturated fatty acid mixtures obtained in the hydrolysis of natural fats and oils, e.g. sunflower oil, soybean oil, olive oil, rapeseed oil, cottonseed oil and tall oil, may also be used.

Suitable non-dimer polycarboxylic acids can have two or more carboxylic acid groups or an equivalent number of anhydride groups on the basis that one anhydride group is equivalent to two acid groups. Such polycarboxylic acids are well known in the art. Preferably the polycarboxylic acid contains two carboxylic acid groups.

Examples of suitable polycarboxylic acids include aliphatic dicarboxylic acids having 2 to 12, preferably 2 to 8 carbon atoms in the alkylene radical. These acids include, for example, aliphatic dicarboxylic acids such as adipic acid, glutaric acid, pimelic acid, suberic acid, azelaic acid, sebacic acid, undecanedoic acid, dodecanadioic acid, succinic or hexanedioic acid; cycloaliphatic acids such as hexahydrophthalic acid and 1,3- and 1,4-cyclohexane dicarboxylic acid; 1,3- and 1,4-unsaturated alkane dioic acids such as fumaric or maleic acids; and aromatic acids such as phthalic acid and terephthalic. The anhydrides of the aforementioned polybasic acids such as maleic anhydride or phthalic anhydride can also be used. A combination of two or more of the polybasic acids may also be used. In one embodiment, it is preferred to use glutaric acid, succinic acid, adipic acid or a combination thereof. Such combination of acids are commercially available and generally comprise from 19 to 26 weight percent adipic acid, from 45-52 weight percent glutaric acid, and 16 to 24 weight percent succinic acid.

Examples of suitable polyhydroxy compounds are ethylene glycol, diethylene glycol, propylene glycol, dipropylene glycol, 1,3-propanediol, 1,10-decanediol, glycerine, trimethylolpropane, 1,4-butanediol, 1,6-hexanediol and 1,3-/1,4-cyclohexanedimethanol. If trifunctional or higher alcohols are used for the manufacture of the polyester polyols, for the production of elastomer for shoe soles, their amount is generally chosen in such that the functionality of a blend is a maximum of 2.8, preferably from 2 to 2.3. In one embodiment, ethylene glycol, diethylene glycol, butanediol, or a combination is used as an additional glycol component.

In addition to the dimer fatty acids, dimerisation usually results in varying amounts of oligomeric fatty acids, such as trimers, and residues of monomeric fatty acids, or esters thereof, being present. Commercially available products, such as those available from Uniqema, generally have a dicarboxylic (dimer) content of greater than 60% and up to greater than 95%. The trimer content is generally less than 40% and is preferably in the range of 2 to 25% for use in the present invention.

The polyester polyol preferably has a molecular weight number average in the range from 1,000 to 5,000, more preferably 1,700 to 3,000, particularly from 1,800 to 2,500 and more preferably from 1,900 to 2,200. The polyester preferably has a hydroxyl number from 10 to 100, preferably from 30 to 80 and more preferably from 40 to 70 mg KOH/g. In addition, the polyester generally has an acid value of less than 2, preferably less than 1.5, and more preferably less than 1.3.

Processes for the production of polyester polyols are well known in the art. To prepare the polyester polyols, the dimer and non-dimer polycarboxylic acids are polycondensed with polyhydroxy compounds. To remove volatile byproducts, the polyester polyols can be subjected to distillation under reduced pressure, stripping with an inert gas, vacuum, etc.

The at least a first polyol composition and the at least a second polyol composition may optionally include another kind of polyol, which includes at least one conventional petroleum-based polyol. Conventional petroleum-based polyols includes materials having at least one group containing an active hydrogen atom capable of undergoing reaction with an isocyanate, and not having parts of the material derived from a vegetable or animal oil. Suitable conventional petroleum-based polyols are well known in the art and include those described herein and any other commercially available polyol. Mixtures of one or more polyols and/or one or more polymer polyols may also be used to produce polyurethane products according to embodiments of the present invention.

Representative conventional petroleum-based polyols include polyether polyols, polyester polyols, polyhydroxy-terminated acetal resins, hydroxyl-terminated amines and polyamines. Alternative polyols that may be used include polyalkylene carbonate-based polyols and polyphosphate-based polyols. Preferred are polyols prepared by adding an alkylene oxide, such as ethylene oxide, propylene oxide, butylene oxide or a combination thereof, to an initiator having from 2 to 8, preferably 2 to 6 active hydrogen atoms. Catalysis for this polymerization can be either anionic or cationic, with catalysts such as KOH, CsOH, boron trifluoride, or a double cyanide complex (DMC) catalyst such as zinc hexacyanocobaltate or quaternary phosphazenium compound. The initiators suitable for the natural oil based polyols may also be suitable for the at least one conventional petroleum-based polyol.

The at least one conventional petroleum-based polyol may for example be poly(propylene oxide) homopolymers, random copolymers of propylene oxide and ethylene oxide in which the poly(ethylene oxide) content is, for example, from about 1 to about 30% by weight, ethylene oxide-capped poly(propylene oxide) polymers and ethylene oxide-capped random copolymers of propylene oxide and ethylene oxide. The polyether polyols may contain low terminal unsaturation (for example, less that 0.02 meq/g or less than 0.01 meq/g), such as those made using so-called double metal cyanide (DMC) catalysts. Polyester polyols typically contain about 2 hydroxyl groups per molecule and have an equivalent weight per hydroxyl group of about 400-1500.

The conventional petroleum-based polyols may be a polymer polyol. In a polymer polyol, polymer particles are dispersed in the conventional petroleum-based polyol. Such particles are widely known in the art an include styrene-acrylonitrile (SAN), acrylonitrile (ACN), polystyrene (PS), methacrylonitrile (MAN), or methyl methacrylate (MMA) particles. In one embodiment the polymer particles are SAN particles.

The conventional petroleum-based polyols may constitute up to about 10 weight %, 20 weight %, 30 weight %, 40 weight %, 50 weight %, or 60 weight % of polyol formulation. The conventional petroleum-based polyols may constitute at least about 1 weight %, 5 weight %, 10 weight %, 20 weight %, 30 weight %, or 50 weight % of polyol formulation.

The at least a first polyol composition and the at least a second polyol composition may optionally also include at least one chain extender. For purposes of the embodiments of the invention, a chain extender is a material having two isocyanate-reactive groups per molecule and an equivalent weight per isocyanate-reactive group of less than 400, preferably less than 300 and especially from 31-125 daltons. Representative of suitable chain-extending agents include polyhydric alcohols, aliphatic diamines including polyoxyalkylenediamines, and mixtures thereof. The isocyanate reactive groups are preferably hydroxyl, primary aliphatic amine or secondary aliphatic amine groups. The chain extenders may be aliphatic or cycloaliphatic, and are exemplified by triols, tetraols, diamines, triamines, aminoalcohols, and the like. Representative chain extenders include ethylene glycol, diethylene glycol, 1,3-propane diol, 1,3- or 1,4-butanediol, dipropylene glycol, 1,2- and 2,3-butylene glycol, 1,6-hexanediol, neopentylglycol, tripropylene glycol, 1,2-ethylhexyldiol, ethylene diamine, 1,4-butylenediamine, 1,6-hexamethylenediamine, 1,5-pentanediol, 1,6-hexanediol, 1,3-cyclohexandiol, 1,4-cyclohexanediol; 1,3-cyclohexane dimethanol, 1,4-cyclohexane dimethanol, N-methylethanolamine, N-methyliso-propylamine, 4-aminocyclohexanol, 1,2-diaminotheane, 1,3-diaminopropane, hexylmethylene diamine, methylene bis(aminocyclohexane), isophorone diamine, 1,3- or 1,4-bis(aminomethyl)cyclohexane, diethylenetriamine, and mixtures or blends thereof. The chain extenders may be used in an amount from about 0.5 to about 20, especially about 2 to about 16 parts by weight per 100 parts by weight of the polyol component.

In addition to the above described polyols, the polyol compositions may also include other ingredients such as catalysts, silicone surfactants, preservatives, and antioxidants.

The at least a first polyol composition and the at least a second polyol composition may optionally also include at least one vegetable oil, such as soy oil, olive oil, canola oil, etc. The vegetable oil may act to dilute and reduce the viscosity of the at least a first polyol composition and/or the at least a second polyol compositions. Furthermore, the vegetable oil may enhance softness of the gel elastomers.

The prepolymer composition may be made by reacting the at least one isocyanate and the at least second polyol composition. Suitable isocyanates for use in preparing the prepolyomer include a wide variety of organic mono- and polyisocyanates. Suitable monoisocyanates include benzyl isocyanate, toluene isocyanate, phenyl isocyanate and alkyl isocyanates in which the alkyl group contains from 1 to 12 carbon atoms. Suitable polyisocyanates include aromatic, cycloaliphatic and aliphatic isocyanates. Exemplary polyisocyanates include m-phenylene diisocyanate, toluene-2-4-diisocyanate, toluene-2-6-diisocyanate, isophorone diisocyanate, 1,3- and/or 1,4-bis(isocyanatomethyl)cyclohexane (including cis- or trans-isomers of either), hexamethylene-1,6-diisocyanate, tetramethylene-1,4-diisocyanate, cyclohexane-1,4-diisocyanate, hexahydrotoluene diisocyanate, methylene bis(cyclohexaneisocyanate) (H₁₂MDI), naphthylene-1,5-diisocyanate, methoxyphenyl-2,4-diisocyanate, diphenylmethane-4,4′-diisocyanate, 4,4′-biphenylene diisocyanate, 3,3′-dimethoxy-4,4′-biphenyl diisocyanate, 3,3′-dimethyl-4-4′-biphenyl diisocyanate, 3,3′-dimethyldiphenyl methane-4,4′-diisocyanate, 4,4′,4″-triphenyl methane triisocyanate, a polymethylene polyphenylisocyanate (PMDI), toluene-2,4,6-triisocyanate and 4,4′-dimethyldiphenylmethane-2,2′,5,5′-tetraisocyanate. In some embodiments, the polyisocyanate is diphenylmethane-4,4′-diisocyanate, diphenylmethane-2,4′-diisocyanate, PMDI, toluene-2,4-diisocyanate, toluene-2,6-diisocyanate or mixtures thereof. Diphenylmethane-4,4′-diisocyanate, diphenylmethane-2,4′-diisocyanate and mixtures thereof are generically referred to as MDI, and all may be used. Toluene-2,4-diisocyanate, toluene-2,6-diisocyanate and mixtures thereof are generically referred to as TDI, and all may be used.

Derivatives of any of the foregoing isocyanate groups that contain biuret, urea, carbodiimide, allophonate and/or isocyanurate groups may also be used. These derivatives often have increased isocyanate functionalities and are desirably used when a more highly crosslinked product is desired.

The proportions of the isocyanate and the at least second polyol composition are chosen to provide an isocyanate terminated prepolymer product. This can be accomplished by using excess stoichiometric amount of polyisocyanate, that is, more than one isocyanate group per active hydrogen group, preferably hydroxyl, amine and unreacted carboxyl group of the at least second polyol composition. The ratio of isocyanate groups to active hydrogen, more preferably hydroxyl and amine groups, on the at least second polyol composition is preferably at least about 1.0, 1.2. 1.4, 1.5, 1.7, or 1.8, and independently preferably at most about 10, preferably at most about 6, preferably at most about 3. Higher (that is stoichiometric amounts or excess) isocyanate levels are optionally used.

Reaction of the at least second polyol composition with the polyisocyanate can be catalyzed using at least one catalyst within the skill in the art for such reactions. Examples of urethane catalysts include tertiary amines such as triethylamine, 1,4-diazabicyclo[2.2.2.]octane (DABCO), N-methylmorpholine, N-ethylmorpholine, N,N,N′,N′-tetramethylhexamethylenediamine, 1,2-dimethylimidazol; and tin compounds such as tin(II)acetate, tin(II)octanoate, tin(II)laurate, dibutyltin dilaurate, dibutyltin dimaleate, dioctyltin diacetate and dibutyltin dichloride. The catalysts are optionally used alone or as mixtures thereof. The reaction may be heated to temperatures between 20° C. and 100° C., and may take 2-6 hours to complete.

The prepolymer composition may optionally also include at least one vegetable oil, such as soy oil, olive oil, canola oil, etc. The vegetable oil may act to dilute and reduce the viscosity of the prepolymer composition. Furthermore, the vegetable oil may enhance softness of the gel elastomers.

The first polyol composition and the prepolymer composition may then be used to form a polyurethane product, such as a gel or soft elastomer. The compositions of the first polyol composition and the second polyol composition of the prepolymer composition may be selected in numerous ways. For example, in one embodiment, all the polyols selected may be a NOBP, that is, the prepolymer may be made by reacting the isocyanate with only NOBPs, and that prepolymer may then be reacted with a poloyol side where all the polyols are NOBPs, which may be the same or different NOBP than was used to make the prepolymer. In an alternative embodiment, one or both of the first or second polyol compositions may also include a conventional petroleum-based polyol, such as a polyether polyol. In certain embodiments, the NOBP used in the first polyol composition may be a NOBP made by reacting the hydroxymethylated monomers with a first initiator, and the second polyol composition may be a NOBP made by reacting the hydroxymethylated monomers with a second initiator. In one embodiment, the first initiator may be an alkoxylated initiator having a functionality of between about 2 and about 4, and the second initiator may be a cycloaliphatic diol (such as UNOXOL). Alternatively, the NOBPs used in the first polyol composition may be a mixture of the first initiator made NOBPs and the second initiator made NOBPs, and/or the NOBPs used in the second polyol composition may be a mixture of the first initiator made NOBPs and the second initiator made NOBPs.

In other embodiments, only one of the first and second polyol compositions may include a NOBP or a blend of different NOBPs.

The first polyol composition may be reacted with the prepolymer compostion at various isocyante indexes. Isocyanate index as used herein is the equivalents of isocyanate, divided by the total equivalents of isocyanate-reactive hydrogen containing materials, multiplied by 100. Considered in another way, it is the ratio of isocyanate-groups over isocyanate-reactive hydrogen atoms present in a formulation, given as a percentage. Thus, the isocyanate index expresses the percentage of isocyanate actually used in a formulation with respect to the amount of isocyanate theoretically required for reacting with the amount of isocyanate-reactive hydrogen used in a formulation.

It is commonly known to use low isocyanate indexes (such as between about 40 and about 80) to make soft gel type elastomers. However, surprisingly, it is possible to obtain soft gel type elastomers by using the NOBPs described herein even at isocyanate indexes as high as at least 100.

In embodiments of the invention, soft gel type elastomers are obtained at indexes as high as 100 when a monol is also included in the first polyol composition. The monol may be a conventional petroleum-based monol, such as a polyether monol, or the monomer used to make NOBP. In one embodiment, the monomer is hydroxymethylated soy bean fatty acids or methyl esters thereof which may have an average OH functionality of between about 0.9 and about 1.1 per fatty acid, preferably, the functionality is about 1. The monol may be present in the first polyol composition in ranges between about 1 and about 70 weight percent of the total weight of the first polyol composition, preferably, between about 5 and 60 weight percent, or, preferably, between about 10 and 55 weight percent.

The gel or soft elastomer-forming reaction, which may proceed slowly as such, may optionally be accelerated by adding catalysts. Suitable catalysts include, for example, tertiary amines such as triethylamine, tributylamine, N-methylmorpholine, N-ethylmorpholine, N,N,N,N-tetramethylethylenediamine, triethylenediamine, 1,4-diaza-bicyclo-(2,2,2)-octane, N-methyl-N-dimethylaminoethylpiperazine, N,N-dimethylbenzylamine, bis-(N,N-diethylaminoethyl)-adipate, pentamethyldiethylenetriamine, N,N-dimethylcyclohexylamine, N,N,N′,N′-tetramethyl-1,3-butanediamine, N,N-dimethyl-β-phenylethylamine, 1,2-dimethylimidazole and 2-methylimidiazole. As catalysts there may also be used Mannich bases known per se and formed from secondary amines such as dimethylamine, and aldehydes, preferably formaldehyde, or ketones such as acetone, methyl ethyl ketone or cyclohexanone, and phenols such as phenol, nonylphenol or bisphenols. Also suitable as catalysts are nitrogen-containing bases such as tetralkyl ammonium hydroxides, alkali metal hydroxides such as sodium hydroxide, alkali phenolates such as sodium phenolate, or alkali metal alcoholates such as sodium methylate. Hexahydrothiazines may also be used as catalysts. In addition, organometallic compounds, in particular organic tin compounds, are also suitable as catalysts. Preferred organic tin compounds are tin(II) salts of carboxylic acids such as tin(II) acetate, octoate, ethylhexoate, and tin(V)-compounds, for example dibutyltin(IV) oxide, chloride, acetate, dilaurate, maleate or dioctyltin acetate.

The gel or soft elastomer-forming reaction components may also include fillers as known in the art. The fillers may include inorganic and/or organic fillers, coloring agents, water-binding agents, surface-active substances, plant protection agents, extenders and/or plasticizers.

Inorganic fillers may for example include: barytes, chalk, gypsum, kieserite, sodium carbonate, titanium oxide, quartz sand, kaolin, carbon black or glass beads. Organic fillers may for example include: powders based on polystyrene, polyvinyl chloride, urea-formaldehyde compositions and/or polyhydrazodicarbonamides (obtained for example from hydrazine and toluoylene diisocyanate). Hollow microspheres of organic origin may also be added.

Inorganic and/or organic fillers may also be used in the form of short fibers. Suitable as short fibers are for example glass fibers and/or fibers of organic origin, for example polyester or polyamide fibers. The short fibers may for example be 0.01 to 1 cm long. Inorganic fillers may also be metal powders, for example iron or copper powder.

As coloring agents the gel or soft elastomer compositions according to embodiments of the invention may for example contain dyes and/or pigments based on organic and/or inorganic compounds and known per se for coloring polyurethane, for example iron oxide and titaniumdioxide.

In a particular embodiment of the production of the gel compositions according to the invention, air or another gas may be forced or stirred into the reaction mixture, for example in an amount of up to 60 vol. %, referred to the gel volume. The gel compositions produced in this way are characterized by a low weight. In some embodiments, water may be added to the system, which in turn reacts with the isocyanate to form CO₂ gas.

In another embodiment of the production of the gel compositions according to the invention, air or any other gas formed or trapped during the reaction may be evacuated using vacuum treatment before mixing and after casting into a mould in order to remove any bubbles caused by stirring as well as dissolved gases from both the first polyol composition and the first prepolymer composition.

The production of articles containing gel or soft elastomer compositions according to embodiments of the invention may be carried out in various ways. The gel or soft elastomer compositions may for example first of all be produced in a mould and the dimensionally stable gel composition resulting from the reaction is then optionally covered, lacquered or coated with a flexible film or a flexible material. Alternatively, the components required for the production of the gel composition are mixed in a mechanical mixer and the mixture is then poured directly into a sheathing of an elastic, flexible film or an elastic coated textile sheet material.

After addition of the mixture the sheathing can be sealed tight and the gel or soft elastomer formation can be left to take place inside by itself. Optionally the sheathing can be inserted between two plane-parallel plates or added to another mould during the gel formation. A gel-containing structural part is then obtained having substantially parallel upper and lower sides or a shape corresponding to the inside of the mould that is used. Depending on the nature of the reaction components, added catalysts and temperature conditions, the time up to the end of the gel formation may for example be from 1 minute up to 12 hours. The temperature of the components that are used is preferably 10 to 70° C.

This procedure enables articles of any arbitrary size and shape to be fabricated in a simple way by producing the sheathings in a generally known manner in the appropriate mold and filling them with the composition that is formed. The thickness of the articles may also be varied within wide limits. When used as seat cushions, generally square in shape with an edge length of 35 to 45 cm, the best results are obtained with thicknesses of more than 2 cm. When used as mattresses, mattress inlays or mattress covers, articles of smaller thickness may also be advantageous.

The gel or soft elastomer compositions according to an embodiment of the invention have the property that they deform under pressure and thus distribute the pressure, i.e. reduce pressure points, and return to their initial state after the deforming force has been removed. This property means that articles containing gel compositions according to the invention are able to change shape to such an extent under the pressure of a person sitting or lying on them that possible pressure wounds are avoided or the healing process of existing wounds is promoted.

Structural parts containing gel or soft elastomer compositions according to the invention may be used in a wide variety of ways, for example as gel cushions in orthopedic shoes and sports shoes, on bicycle saddles, under horse riding saddles, on wheelchairs and sickbeds, on seating surfaces, back-supporting surfaces, headrests and armrests of chairs, car seats or other types of seating, on operating tables or medical examination tables, or in incubators. Optionally, the gels or soft elastomers may be combined with flexible foams for use in for example wheel chair seats, bicycle seats, etc.

Furthermore, structural parts or articles consisting of a gel composition according to the invention may be used as supports and coverings on elbows, shin bones or foot surfaces in order to avoid and reduce the effects of injuries, especially sports injuries, as supports for cosmetic masks, for example face masks, as self-adhesive coverings, for purposes of securement, for bandages and dressings for eyes and ears; as a support for slack breast tissue, as a cushion underneath horse saddles, on prostheses or on diapers to reduce pressure points. The gel compositions according to the invention may be used for prosthesis supports (gel or polyurethane liners) that are wound over the stump. They may furthermore be used for bicycle saddles and shoe inserts. Another possible application is in instrument panels and dashboards that are to be provided with a soft-touch feel. For this purpose films and/or textiles are coated with the gel according to the invention.

In yet another embodiment, the soft PU-gels according to embodiments of the invention show excellent self-adhesion to various surfaces such as concrete, asphalt, cement etc. Typically the level of self-adhesion of the PU-gels increases with the softness of the elastomers. Due to the excellent pressure sensitive adhesion to surfaces, soft PU-gels can be used as sealing mats to protect against environmental problems, which may occur during leakages etc. Such sealing mats may be used to seal manhole covers to protect the environment against liquid pollution, i.e. occurrence of spillages during the unloading of tank trucks, intrusion of extinguishing water into the sewer system during firefighting measures etc. The sealing mats may not only be used in case of leakages, but also in preventive sealing applications.

Alternatively sealing mats with layers having different hardness (sticky side and non sticky side) are used.

In one embodiment, the sealing mat may contain one PU-gel layer covered with 2 different foils. The foil on top (preferably transparent, but also standard opaque foils are possible) adheres permanently to the gel, the foil on the bottom is removable. In use, the non-adhesive foil is removed and the transparent gel side will be placed on the sealing object. The key performance characteristic for this application is good stickiness. After use the mats can be washed to remove dust without losing the tack of the gel. In case of exposure to chemicals, the mats may undergo testing before they can be reused again. The mats have also been proven to be tolerant to exposure to load exerted by foot or vehicle traffic.

The gel or soft elastomer compositions according to the invention exhibit an improvement in chemical properties, such as chemical resistance to water, aqueous solutions of sodium chloride, aqueous solutions of soaps and detergents, aqueous solutions of peroxides, basic solutions, acidic solutions, and various organic solvents such as toluene, DMF, ethylacetate, acetone, ethanol, and methanol.

The gel or soft elastomer compositions according to the invention may exhibit Shore 00 hardness values (measured according to ASTM D 2240) of between about 0 and about 80, although higher values are obtainable as well. Medium soft elastomers or gels may exhibit Shore 00 hardness values of between about 70 and about 80. Soft elastomers or gels may exhibit Shore 00 hardness values of between about 50 and about 70. Extra soft elastomers or gels may exhibit Shore 00 hardness values of between about 0 and about 50. In certain embodiments the Shore 00 hardness values are between about 0, 1, 3, 7, 9, 10, 12, 14, 15, 20, or 25 and about 26, 28, 30, 32, 34, 35, 37, 39, 40, 43, 45, 47, 50, 55, 60, 65, or 80.

### EXAMPLES

The following examples are provided to illustrate the embodiments of the invention, but are not intended to limit the scope thereof. All parts and percentages are by weight unless otherwise indicated.

The following materials were used:
Monomer Hydroxymethylated fatty acid methyl ester monomers with an average of 1.0 hydroxyls per fatty acid derived from soy oil in its natural abundance made according to U.S. Pat. No. 7,615,658.
NOBP A A nominally 2.0-functional natural oil polyol prepared by reacting Monomer with an approximately 50/50% weight mixture of 1,3-cyclohexane dimethanol and 1,4-cyclohexane dimethanol (commercially available from The Dow Chemical Company under the trade designation Unoxol™), using 650 ppm stannous octoate (commercially available from City Chemical Co.) as the catalyst. NOBP-A has an average of approximately 2.0 hydroxyl groups/molecule, an OH number of 52.9, and number average molecular weight of about 2120.
NOBP B A nominally 2.0-functional natural oil polyol prepared by reacting Monomer with an approximately 50/50% weight mixture of 1,3-cyclohexane dimethanol and 1,4-cyclohexane dimethanol (commercially available from The Dow Chemical Company under the trade designation Unoxol™), using 650 ppm stannous octoate (commercially available from City Chemical Co.) as the catalyst. NOBP-A has an average of approximately 2.0 hydroxyl groups/molecule, an OH number of 73.3, and number average molecular weight of about 1530.
NOBP C Soybean oil based polyol prepared according to examples 19-22 of WO 2004/096882. The monomers are hydroxymethylated soybean fatty acid methyl esters and the initiator is a 625 molecular weight poly(ethylene oxide) triol used at a ratio of monomer to initiator of 4.1:1. The polyol has an OH number of 89.
ISONATE* 50 OP A 50 percent 4,4′-methylene diphenyl isocyanate, 50 percent 2,4′-methylene diphenyl isocyanate mixture having a functionality of 2.0 and an equivalent weight of 125 g/equivalent available from The Dow Chemical Company.
VORASTAR* HB 6002 A prepolymer based on MDI (50 percent 4,4′-methylene diphenyl isocyanate, 50 percent 2,4′-methylene diphenyl isocyanate) and polyether diols (NCO content of 10%), available from The Dow Chemical Company.
VORASTAR* HB 6013 A prepolymer based on MDI (70 percent 4,4′-methylene diphenyl isocyanate, 30 percent 2,4′-methylene diphenyl isocyanate) and polyether diols (NCO content of 10%), available from The Dow Chemical Company.
VORASTAR* HA 6057 A monofunctional polyetherpolyol with OH-number about 27, number average molecular weight of about MW 2000, and an EO/PO ratio of 15/85, available from The Dow Chemical Company.
VORALUX* HF 505 A sorbitol initiated polyol having a hydroxyl number of 29 and a number average molecular weight of about 12000, available from The Dow Chemical Company.
VORANOL* CP 6001 A glycerol initiated polyoxypropylene polyoxyethylene polyol having an average hydroxyl number of 28 and a number average molecular weight of about 6000, available from The Dow Chemical Company.
Monoethylene glycol Available from The Dow Chemical Company.
Dipropylene glycol Available from The Dow Chemical Company.
Benzoyl Chloride Available from Moeller Chemie.
DABCO 33-LV: A 33% solution of triethylenediamine in propylene glycol available from Air Products & Chemicals Inc.
BYK 066 N Solution of foam destroying polysiloxanes. Available from BYK Additives & Instruments.
   * ISONATE, VORALUX, VORANOL, and VORASTAR are trademarks of The Dow Chemical Company.

The following test methods were used:
The hardness (Shore 00) was measured according to ASTM D 2240, Test Method for Rubber Property—Durometer Hardness. The higher the value, the harder the elastomer. Samples had a thickness of at minimum 0.8-1 cm. For some samples, a hardness range is listed. This range reflects observations made during the measurement of Shore00 (The hardness was changing). The high number was directly recorded after the Shore00 clock was set on the sample. The lower number represents the endpoint when the Shore00 hardness movement stopped.
The Isocyanate content was measured by the dibutylamine titration method according to ASTM D 5155
The determination of polyol hydroxyl number was measured by a titration method according to ASTM D 4274.
The measurement of water content in polyols was performed using the Karl Fischer titration method according to ASTM E 203.
The viscosity of polyols and isocyanates were measured according to DIN 53019

### Prepolymer Preparation

NOBP based preopolymers (Prepol A and B) were prepared by a controlled reaction of an excess of the isocyanates with the NOBP. A reaction vessel equipped with chemicals addition inlet, heating mantle, electrical stirrer, thermometer, and gas inlet and outlet for continuous flow of nitrogen, was charged with the isocyanate. The reaction was performed by stirring the isocyanate compounds and the benzoyl chloride and feeding the NOBP into the reaction vessel at a controlled rate over about 2 hours, while maintaining the temperature in the vessel at about 60-75° C. After a total reaction time of about 3 hours, the isocyanate content was at the theoretical value. The Prepolymer was unloaded after stopping the reaction by cooling.

TABLE 1

| | Prepol-A | Prepol-B |
|---|---|---|
| ISONATE OP 50 | 37.34 | 39.72 |
| Benzoyl chloride | 0.02 | 0.02 |
| NOBP A | 62.74 | |
| NOBP B | | 60.26 |

Prepolymer Properties

| | | |
|---|---|---|
| NCO % | 9.7 | 9.9 |
| Viscosity 23° C. (mPas) | 8800 | 12000 |

### Gel/Soft Elastomer Preparation

Room temperature processing conditions were used for the PU gel samples prepared for this work. The polyol and isocyanate component were mixed together in the calculated mixing ratios listed in the following tables. In order to manufacture bubble-free material the elastomer samples required a special manufacturing procedure: vacuum treatment before mixing and after casting into a mould was used to remove the bubbles caused by stirring as well as dissolved gases from both components. The samples were manufactured by casting on non-adhesive substrates at room temperature.

### Examples 1-28

### NOBP and Prepolymer

Elastomers using only polyols that are NOBP polyols are made in Examples 1-28. That is, NOBP polyols are used on as polyol (or blend of polyols) and in the prepolymer.

TABLE 2

| | Example | | | | | | | | | |
|---|---|---|---|---|---|---|---|---|---|---|
| | 1 | 2 | 3 | 4 | 5 | 6 | 7 | 8 | 9 | 10 |
| NOBP C | 100 | 100 | 100 | 100 | 100 | 100 | 100 | 100 | 100 | 100 |
| Dabco 33 | 0.4 | 0.4 | 0.4 | 0.4 | 0.4 | 0.4 | 0.4 | 0.4 | 0.4 | 0.4 |
| LV | | | | | | | | | | |
| Byk 066 | 0.1 | 0.1 | 0.1 | 0.1 | 0.1 | 0.1 | 0.1 | 0.1 | 0.1 | 0.1 |

Prepol A

| | | | | | | | | | | |
|---|---|---|---|---|---|---|---|---|---|---|
| Index | 100 | 90 | 80 | 75 | 70 | 65 | 60 | 55 | 50 | 45 |
| Mixing | 73.3 | 66.0 | 58.7 | 55.0 | 51.3 | 47.7 | 44.0 | 40.3 | 36.7 | 33.0 |
| ratio | | | | | | | | | | |
| Pol.:Iso. = | | | | | | | | | | |
| 100:x | | | | | | | | | | |
| Hardness | 80 | 80 | 76 | 73 | 70 | 65 | 54 | 43-39 | 25-15 | 8-0 |
| (Shore 00) | | | | | | | | | | |
| Appearance | milky | milky | milky | milky | milky | milky | milky | milky | milky | milky |
| (Visual) | opaque | opaque | opaque | opaque | opaque | opaque | opaque | opaque | opaque | opaque |
| Appearance | brittle | brittle | brittle | brittle | slightly | elastic | elastic | elastic | elastic | elastic |
| (Elasticity) | | | | | brittle | | | | | |

TABLE 3

| | Example | | | | | | |
|---|---|---|---|---|---|---|---|
| | 11 | 12 | 13 | 14 | 15 | 16 | 17 |
| NOBPC | 75 | 75 | 75 | 75 | 75 | 75 | 75 |
| NOBP A | 25 | 25 | 25 | 25 | 25 | 25 | 25 |
| Dabco 33 LV | 0.1 | 0.1 | 0.1 | 0.3 | 0.3 | 0.3 | 0.3 |
| Byk 066 | 0.1 | 0.1 | 0.1 | 0.1 | 0.1 | 0.1 | 0.1 |

Prepol-A

| | | | | | | | |
|---|---|---|---|---|---|---|---|
| Index | 70 | 65 | 60 | 55 | 50 | 45 | 40 |
| Mixing ratio | 46.0 | 42.7 | 39.4 | 35.8 | 32.6 | 29.3 | 26.1 |
| Pol.:Iso. = 100:x | | | | | | | |
| Hardness (Shore 00) | 64 | 58 | 52 | 35 | 30-22 | 5-0 | liquid |
| Appearance (Visual) | milky | milky | milky | milky | milky | milky | milky |
| | opaque | opaque | opaque | opaque | opaque | opaque | opaque |
| Appearance (Elasticity) | elastic | elastic | elastic | elastic | elastic | elastic | n.m. |

TABLE 4

| | Example | | | | | |
|---|---|---|---|---|---|---|
| | 18 | 19 | 20 | 21 | 22 | 23 |
| NOBP C | 100 | 100 | 100 | 100 | 100 | 100 |
| Dabco 33 LV | 0.1 | 0.1 | 0.1 | 0.1 | 0.1 | 0.1 |
| Byk 066 | 0.1 | 0.1 | 0.1 | 0.1 | 0.1 | 0.1 |

Prepol-B

| | | | | | | |
|---|---|---|---|---|---|---|
| Index | 65 | 60 | 55 | 50 | 45 | 40 |
| Mixing ratio | 46.5 | 42.9 | 39.3 | 35.7 | 32.2 | 28.6 |
| Pol.:Iso. = | | | | | | |
| 100:x | | | | | | |
| Hardness | 68 | 60 | 41 | 20 | 5-0 | 0 |
| (Shore 00) | | | | | | |
| Appearance | milky | milky | milky | milky | milky | milky |
| (Visual) | opaque | opaque | opaque | opaque | opaque | opaque |
| | | e | | | | |
| Appearance | elastic | elastic | elastic | elastic | elastic | elastic |
| (Elasticity) | | | | | | |

TABLE 5

| Example | 24 | 25 | 26 | 27 | 28 |
|---|---|---|---|---|---|
| NOBP C | 75 | 75 | 75 | 75 | 75 |
| NOBP A | 25 | 25 | 25 | 25 | 25 |
| Dabco 33 LV | 0.1 | 0.1 | 0.1 | 0.3 | 0.3 |
| Byk 066 | 0.1 | 0.1 | 0.1 | 0.1 | 0.1 |

Prepol-B

| | | | | | |
|---|---|---|---|---|---|
| Index | 75 | 70 | 65 | 60 | 55 |
| Mixing ratio | 48.0 | 44.8 | 41.6 | 38.4 | 35.2 |
| Pol.:Iso. = 100:x | | | | | |
| Hardness (Shore 00) | 71 | 65 | 57 | 48 | 36 |
| Appearance (Visual) | milky | milky | milky | milky | milky |
| | opaque | opaque | opaque | opaque | opaque |
| Appearance (Elasticity) | elastic | elastic | elastic | elastic | elastic |

As can be seen from the data above it is possible to adapt a low isocyanate index technology to obtain soft elastomer systems fully formulated from NOBP's.

### Examples 29-35

### NOBP and Polyether Based Prepolymer

Examples 29-35 are elastomers made using a system where NOBP polyols are used as the polyol and polyether based polyols are used in the prepolymer.

TABLE 6

| | Example | | | | | | |
|---|---|---|---|---|---|---|---|
| | 29 | 30 | 31 | 32 | 33 | 34 | 35 |
| NOBP C | 100 | 100 | 100 | 100 | 100 | 100 | 100 |
| Dabco 33 LV | 0.2 | 0.2 | 0.2 | 0.2 | 0.2 | 0.2 | 0.2 |
| Byk 066 | 0.1 | 0.1 | 0.1 | 0.1 | 0.1 | 0.1 | 0.1 |

VORASTAR HB 6002

| | | | | | | | |
|---|---|---|---|---|---|---|---|
| Index | 75 | 70 | 65 | 60 | 55 | 50 | 45 |
| Mixing ratio | 52.7 | 49.2 | 45.7 | 42.2 | 38.7 | 35.2 | 31.6 |
| Pol.:Iso. = 100:x | | | | | | | |
| Hardness (Shore 00) | 78 | 74 | 68 | 54 | 40-33 | 20-5 | 0 |
| Appearance (Visual) | clear | clear | clear | clear | clear | clear | clear |
| | transparent | transparent | transparent | transparent | transparent | transparent | transparent |
| | yellowish | yellowish | yellowish | yellowish | yellowish | yellowish | yellowish |
| Appearance (Elasticity) | slightly | slightly | elastic | elastic | elastic | elastic | n.m. |
| | elastic | elastic | | | | | |

### Examples 36-49

### NOBP/Polyether Based Monol and Polyether Based Prepolymer

Examples 36-49 are elastomers made using a system where NOBP polyols and polyether based monols are used as the polyol and polyether based polyols are used in the prepolymer.

TABLE 7

| | Example | | | | | | | | | | | | | |
|---|---|---|---|---|---|---|---|---|---|---|---|---|---|---|
| | 36 | 37 | 38 | 39 | 40 | 41 | 42 | 43 | 44 | 45 | 46 | 47 | 48 | 49 |
| NOBP C | 90 | 85 | 80 | 75 | 70 | 65 | 60 | 55 | 50 | 45 | 40 | 35 | 30 | 27 |
| VORASTAR | 10 | 15 | 20 | 25 | 30 | 35 | 40 | 45 | 50 | 55 | 60 | 65 | 70 | 73 |
| HA 6057 | | | | | | | | | | | | | | |
| polyol | | | | | | | | | | | | | | |
| Dabco 33 LV | 0.3 | 0.3 | 0.3 | 0.3 | 0.3 | 0.3 | 0.3 | 0.3 | 0.4 | 0.4 | 0.4 | 0.4 | 0.4 | 0.4 |
| Byk 066 | 0.1 | 0.1 | 0.1 | 0.1 | 0.1 | 0.1 | 0.1 | 0.1 | 0.1 | 0.1 | 0.1 | 0.1 | 0.1 | 0.1 |

VORASTAR HB 6002

| | | | | | | | | | | | | | | |
|---|---|---|---|---|---|---|---|---|---|---|---|---|---|---|
| Index | 100 | 100 | 100 | 100 | 100 | 100 | 100 | 100 | 100 | 100 | 100 | 100 | 100 | 100 |
| Mixing ratio | 65.8 | 63.4 | 61.0 | 58.5 | 56.1 | 53.7 | 51.2 | 48.8 | 46.4 | 44.3 | 41.9 | 39.5 | 37.0 | 36.7 |
| Pol.:Iso. = | | | | | | | | | | | | | | |
| 100:x | | | | | | | | | | | | | | |
| Hardness | 83 | 82 | 81 | 80 | 77 | 74 | 69 | 63 | 61 | 62 | 53 | 39 | 22 | 10 |
| (Shore 00) | | | | | | | | | | | | | | |
| Appearance | clear | clear | clear | clear | clear | clear | clear | clear | clear | clear | clear | clear | clear | clear |
| (Visual) | trans- | trans- | trans- | trans- | trans- | trans- | trans- | trans- | trans- | trans- | trans- | trans- | trans- | trans- |
| | parent | parent | parent | parent | parent | parent | parent | parent | parent | parent | parent | parent | parent | parent |
| | yellow | yellow | yellow | yellow | yellow | yellow | yellow | yellow | yellow | yellow | yellow | yellow | yellow | yellow |
| Appearance | brittle | brittle | brittle | brittle | brittle | brittle | elastic | Elastic | elastic | elastic | elastic | elastic | elastic | elastic |
| (Elasticity) | | | | | | | | | | | | | | |

It can be seen from the results in Table 7 that it is possible to obtain elastic and soft elastomers by incorporating monofunctional polyols, even at an isocyanate index of 100.

### Examples 50-62

### NOBP/Polyether Based Monol and NOBP Based Prepolymer

Examples 50-62 are elastomers made using a system where NOBP polyols and polyether based monols are used as the polyol and NOBPs are used in the prepolymer.

TABLE 8

| | Example | | | | | | | | | | | | |
|---|---|---|---|---|---|---|---|---|---|---|---|---|---|
| | 50 | 51 | 52 | 53 | 54 | 55 | 56 | 57 | 58 | 59 | 60 | 61 | 62 |
| NOBP C | 90 | 85 | 80 | 75 | 70 | 65 | 60 | 55 | 50 | 45 | 40 | 35 | 30 |
| VORASTAR | 10 | 15 | 20 | 25 | 30 | 35 | 40 | 45 | 50 | 55 | 60 | 65 | 70 |
| HA 6057 | | | | | | | | | | | | | |
| polyol | | | | | | | | | | | | | |
| Dabco 33 | 0.3 | 0.3 | 0.3 | 0.3 | 0.3 | 0.3 | 0.3 | 0.3 | 0.3 | 0.3 | 0.3 | 0.3 | 0.3 |
| LV | | | | | | | | | | | | | |
| Byk 066 | 0.1 | 0.1 | 0.1 | 0.1 | 0.1 | 0.1 | 0.1 | 0.1 | 0.1 | 0.1 | 0.1 | 0.1 | 0.1 |

Prepol-A

| | | | | | | | | | | | | | |
|---|---|---|---|---|---|---|---|---|---|---|---|---|---|
| Index | 100 | 100 | 100 | 100 | 100 | 100 | 100 | 100 | 100 | 100 | 100 | 100 | 100 |
| Mixing | 67.8 | 65.3 | 62.8 | 60.3 | 57.8 | 55.3 | 52.8 | 50.3 | 47.8 | 45.7 | 43.2 | 40.7 | 38.2 |
| ratio | | | | | | | | | | | | | |
| Pol.:Iso. = | | | | | | | | | | | | | |
| 100:x | | | | | | | | | | | | | |
| Hardness | 81 | 79 | 78 | 78 | 74 | 73 | 69 | 66 | 64 | 63 | 57 | 47 | 27 |
| (Shore 00) | | | | | | | | | | | | | |
| Appearance | Milky | Milky | Milky | Milky | Milky | Milky | Milky | Milky | Milky | Milky | Milky | Milky | Milky |
| (Visual) | Opaque | Opaque | Opaque | Opaque | Opaque | Opaque | Opaque | Opaque | Opaque | Opaque | Opaque | Opaque | Opaque |
| Appearance | brittle | brittle | brittle | brittle | brittle | brittle | brittle | Brittle | brittle | elastic | elastic | elastic | elastic |
| (Elasticity) | | | | | | | | | | | | | |

It can be seen from the results in Table 8 that it is possible to obtain elastic and soft elastomers by incorporating monofunctional polyols with an NOBP and using NOBP based preopolymers, even at an isocyanate index of 100.

### Examples 63-70

### Polyether Based Polyol/Polyether Based Monol and NOBP Based Prepolymer

Examples 63-70 are elastomers made using a system where polyether based polyols and polyether based monols are used as the polyol and NOBPs are used in the prepolymer.

TABLE 9

| | Example | | | | | | | |
|---|---|---|---|---|---|---|---|---|
| | 63 | 64 | 65 | 66 | 67 | 68 | 69 | 70 |
| VORANOL CP | 100 | 90 | 80 | 75 | 70 | 65 | 63 | 61 |
| 6001 polyol | | | | | | | | |
| VORASTAR HA | 0 | 10 | 20 | 25 | 30 | 35 | 37 | 39 |
| 6057 polyol | | | | | | | | |
| Dabco 33 LV | 0.3 | 0.3 | 0.3 | 0.3 | 0.3 | 0.3 | 0.4 | 0.4 |
| Byk 066 | 0.1 | 0.1 | 0.1 | 0.1 | 0.1 | 0.1 | 0.1 | 0.1 |

Prepol-A

| | | | | | | | | |
|---|---|---|---|---|---|---|---|---|
| Index | 100 | 100 | 100 | 100 | 100 | 100 | 100 | 100 |
| Mixing ratio | 22.8 | 22.8 | 22.8 | 22.8 | 22.8 | 22.8 | 23.2 | 23.2 |
| Pol.:Iso. = 100:x | | | | | | | | |
| Hardness | 70-68 | 66-62 | 55-50 | 50-44 | 38-28 | 30-18 | 20-12 | 20-5 |
| (Shore 00) | | | | | | | | |
| Appearance | opaque | opaque | opaque | opaque | opaque | opaque | opaque | Opaque |
| (Visual) | | | | | | | | |
| Apperance | elastic | elastic | elastic | elastic | elastic | elastic | elastic | Elastic |
| Elasticity) | | | | | | | | |

It can be seen from the results in Table 9 that it is possible to obtain elastic and soft elastomers by incorporating monofunctional polyols and using NOBP based preopolymers, even at an isocyanate index of 100.

### Comparative Examples A-D

### Polyether Based Polyol/Polyether Based Monol and Polyether Based Prepolymer

Comparative Examples A-D are elastomers made using a system where polyether based polyols and polyether based monols are used as the polyol and polyether based polyols are used in the prepolymer.

TABLE 10

| Comparative Example | A | B | C | D |
|---|---|---|---|---|
| VORANOL CP | 90 | 80 | 70 | 65 |
| 6001 polyol | | | | |
| VORASTAR HA | 10 | 20 | 30 | 35 |
| 6057 polyol | | | | |
| Dabco 33 LV | 0.5 | 0.4 | 0.4 | 0.4 |
| Byk 066 | 0.1 | 0.1 | 0.1 | 0.1 |

VORASTAR HB 6002

| | | | | |
|---|---|---|---|---|
| Index | 100 | 100 | 100 | 100 |
| Mixing ratio | 22.9 | 22.9 | 22.9 | 22.9 |
| Pol.:Iso. = 100:x | | | | |
| Hardness (Shore 00) | 60 | 42 | 12 | 0 |
| Appearance (Visual) | clear | clear | clear | clear |
| | trans- | trans- | trans- | trans- |
| | parent | parent | parent | parent |
| | slightly | slightly | slightly | slightly |
| | yellow | yellow | yellow | yellow |
| Appearance | elastic | elastic | elastic | elastic |
| (Elasticity) | | | | |

### Examples 71-79

### NOBP/Natural Oil Based Monol and Polyether Based Prepolymer

Examples 71-79 are elastomers made using a system where NOBPs and natural oil based monols are used as the polyol and polyether based polyols are used in the prepolymer.

TABLE 11

| | Example | | | | | | | | |
|---|---|---|---|---|---|---|---|---|---|
| | 71 | 72 | 73 | 74 | 75 | 76 | 77 | 78 | 79 |
| NOBP C | 90 | 80- | 75 | 70 | 65 | 60 | 55 | 50 | 48 |
| Monomer | 10 | 20 | 25 | 30 | 35 | 40 | 45 | 50 | 52 |
| Dabco 33 LV | 0.3 | 0.3 | 0.4 | 0.3 | 0.4 | 0.3 | 0.4 | 0.3 | 0.4 |
| Byk 066 | 0.1 | 0.1 | 0.1 | 0.1 | 0.1 | 0.1 | 0.1 | 0.1 | 0.1 |

VORASTAR HB 6002

| | | | | | | | | | |
|---|---|---|---|---|---|---|---|---|---|
| Index | 100 | 100 | 100 | 100 | 100 | 100 | 100 | 100 | 100 |
| Mixing ratio | 76.6 | 82.5 | 85.8 | 88.5 | 91.8 | 94.4 | 97.7 | 100.4 | 101.9 |
| Pol.:Iso. = 100:x | | | | | | | | | |
| Hardness | 80 | 73 | 63 | 57 | 47 | 30 | 15 | 10 | 0 |
| (Shore 00) | | | | | | | | | |
| Appearance | clear | clear | clear | clear | clear | clear | clear | clear | clear |
| (Visual) | transparent | transparent | transparent | transparent | transparent | transparent | transparent | transparent | transparent |
| | slightly | slightly | slightly | slightly | slightly | slightly | slightly | slightly | slightly |
| | yellow | yellow | yellow | yellow | yellow | yellow | yellow | yellow | yellow |
| Appearance | elastic | elastic | elastic | elastic | elastic | elastic | elastic | elastic | elastic |
| (Elasticity) | | | | | | | | | |

It can be seen from the results in Table 11 that it is possible to obtain elastic and soft elastomers by incorporating monofunctional natural oil based monomers with the NOBP and using polyether based preopolymers, at an isocyanate index of 100.

### Examples 80-90

### NOBP/Natural Oil Based Monol and NOBP Based Prepolymer

Examples 80-90 are elastomers made using a system where NOBPs and natural oil based monols are used as the polyol and NOBP based polyols are used in the prepolymer.

TABLE 12

| | Example | | | | | | | | | | |
|---|---|---|---|---|---|---|---|---|---|---|---|
| | 80 | 81 | 82 | 83 | 84 | 85 | 86 | 87 | 88 | 89 | 90 |
| NOBP C | 90 | 80 | 75 | 70 | 65 | 60 | 55 | 50 | 48 | 45 | 40 |
| Monomer | 10 | 20 | 25 | 30 | 35 | 40 | 45 | 50 | 52 | 55 | 60 |
| Dabco 33 LV | 0.3 | 0.3 | 0.3 | 0.3 | 0.3 | 0.3 | 0.3 | 0.3 | 0.3 | 0.3 | 0.3 |
| Byk 066 | 0.1 | 0.1 | 0.1 | 0.1 | 0.1 | 0.1 | 0.1 | 0.1 | 0.1 | 0.1 | 0.1 |

Prepol-A

| | | | | | | | | | | | |
|---|---|---|---|---|---|---|---|---|---|---|---|
| Index | 100 | 100 | 100 | 100 | 100 | 100 | 100 | 100 | 100 | 100 | 100 |
| Mixing ratio | 78.9 | 85.1 | 88.5 | 91.2 | 94.6 | 97.4 | 100.8 | 103.5 | 105.1 | 106.6 | 109.7 |
| Pol.:Iso. = 100:x | | | | | | | | | | | |
| Hardness (Shore 00) | 80 | 76 | 70 | 69 | 62 | 62 | 45 | 38 | 35 | 30 | 7 |
| Appearance | slightly | slightly | slightly | slightly | slightly | slightly | slightly | slightly | slightly | slightly | slightly |
| (Visual) | opaque | opaque | opaque | opaque | opaque | opaque | opaque | opaque | opaque | opaque | opaque |
| Apperance | brittle | brittle | brittle | a bit | a bit | a bit | elastic | elastic | elastic | elastic | elastic |
| (Elasticity) | | | | elastic | elastic | elastic | | | | | |

It can be seen from the results in Table 11 that it is possible to obtain elastic and soft elastomers by incorporating monofunctional natural oil based monomers with the NOBP and using polyether based preopolymers, at an isocyanate index of 100.

### Examples 91-96

### Polyether Based Polyol/Natural Oil Based Monol and Polyether Based Prepolymer

Examples 1-96 are elastomers made using a system where polyether based polyols and natural oil based monols are used as the polyol and polyether based polyols are used in the prepolymer. It can be seen from the results in Table 13 that it is possible to obtain elastic and soft elastomers by incorporating monofunctional natural oil based monomers with the polyether based polyols and using polyether based preopolymers, at an isocyanate index of 100.

TABLE 13

| | Example | | | | | | |
|---|---|---|---|---|---|---|---|
| | E* | 91 | 92 | 93 | 94 | 95 | 96 |
| VORALUX | 100 | 90 | 80 | 75 | 70 | 65 | 60 |
| HF 505 | | | | | | | |
| Monomer | 0 | 10 | 20 | 25 | 30 | 35 | 40 |
| Dabco 33 LV | 0.3 | 0.3 | 0.3 | 0.3 | 0.3 | 0.3 | 0.3 |
| Byk 066 | 0.1 | 0.1 | 0.1 | 0.1 | 0.1 | 0.1 | 0.1 |

VORASTAR HB 6002

| | | | | | | | |
|---|---|---|---|---|---|---|---|
| Index | 100 | 100 | 100 | 100 | 100 | 100 | 100 |
| Mixing | 22.1 | 32.9 | 43.7 | 49.1 | 54.6 | 60.0 | 65.4 |
| ratio | | | | | | | |
| Pol.:Iso. = | | | | | | | |
| 100:x | | | | | | | |
| Hardness | 80 | 66 | 42 | 27 | 10 | 0 | liquid |
| (Shore 00) | | | | | | | |
| Appearance | clear | clear | clear | clear | clear | clear | clear |
| (Visual) | transparent | transparent | transparent | transparent | transparent | transparent | transparent |
| | slightly | slightly | slightly | slightly | slightly | slightly | slightly |
| | yellow | yellow | yellow | yellow | yellow | yellow | yellow |
| Appearance | elastic | elastic | elastic | elastic | elastic | elastic | not |
| (Elasticity) | | | | | | | measured |

*Comparative Example

### Examples 97-104

### Polyether Based Polyol/Natural Oil Based Monol and NOBP Based Prepolymer

Examples 97-104 are elastomers made using a system where polyether based polyols and natural oil based monols are used as the polyol and NOBP based polyols are used in the prepolymer.

TABLE 14

| | Example | | | | | | | | |
|---|---|---|---|---|---|---|---|---|---|
| | F* | 97 | 98 | 99 | 100 | 101 | 102 | 103 | 104 |
| VORALUX HF | 100 | 90 | 80 | 75 | 70 | 65 | 60 | 55 | 50 |
| 505 polyol | | | | | | | | | |
| Monomer | 0 | 10 | 20 | 25 | 30 | 35 | 40 | 45 | 50 |
| Dabco 33 LV | 0.3 | 0.3 | 0.3 | 0.3 | 0.3 | 0.3 | 0.3 | 0.3 | 0.3 |
| Byk 066 | 0.1 | 0.1 | 0.1 | 0.1 | 0.1 | 0.1 | 0.1 | 0.1 | 0.1 |

Prepol-A

| | | | | | | | | | |
|---|---|---|---|---|---|---|---|---|---|
| Index | 100 | 100 | 100 | 100 | 100 | 100 | 100 | 100 | 100 |
| Mixing ratio | 22.8 | 33.9 | 45.1 | 50.7 | 56.2 | 61.8 | 67.4 | 73.0 | 78.5 |
| Pol.:Iso. = 100:x | | | | | | | | | |
| Hardness | 80 | 65 | 52 | 46 | 38 | 25 | 13 | 7 | 0 |
| (Shore 00) | | | | | | | | | |
| Appearance | opaque | opaque | opaque | opaque | opaque | opaque | opaque | opaque | opaque |
| (Visual) | | | | | | | | | |
| Apperance | brittle | brittle | brittle | a bit | a bit | a bit | elastic | elastic | elastic |
| (Elasticity) | | | | elastic | elastic | elastic | | | |

*Comparative Example

### Chemical Resistance

To measure the chemical resistance, gel specimens of Examples 105 and 106 and Comparative Example G were prepared and conditioned in a climate controlled room (about 20° C.; at about 50% relative humidity) (see Table 15 for compositions).

TABLE 15

| Example | G* | 105 | 106 |
|---|---|---|---|
| VORALUX HF 505 polyol | 39.15 | — | — |
| VORASTAR HA 6057 polyol | 34.75 | — | — |
| VORANOL EP 1900 | 23.48 | — | — |
| Monoethylene glycol | 0.75 | — | — |
| Dipropylene glycol | 1.27 | — | — |
| Dabco 33 LV | 0.5 | 0.4 | 0.3 |
| NOBP C | — | 64.68 | 54.78 |
| Monomer | — | 34.82 | 44.82 |
| Byk 066 | 0.1 | 0.1 | 0.1 |
| OH-Number of polyol side | 54.5 | 122.6 | 130.1 |
| Isocyanate | VORASTAR | VORASTAR | Propol-A |
| | HB 6002 | HB 6002 | |
| Index | 100 | 100 | 100 |
| Mixing ratio | 38.0 | 91.8 | 100.8 |
| Pol.:Iso. = 100:x | | | |

*Comparative Example

The weights of the test specimens were recorded before the immersion into various media. Chemical resistance was determined according to ASTM 543-95 test protocol. Specimens were immersed in various media for a period of 3 and 24 h at room temperature. This timeframe was considered as a common range for sealing mats made of soft PU-gel elastomers. Upon completion of the respective time intervals, the immersed samples were removed from the test container and the specimens were weighed. The increase in weight was calculated according to the following formula:

Weight gain[%]=100×((wet weight−conditioned weight)/conditioned weight)

TABLE 16

| | Weight gain after | | | | | | Weight gain after | | | | | |
|---|---|---|---|---|---|---|---|---|---|---|---|---|
| | 3 h [%] | | | Sample appearance | | | 24 h [%] | | | Sample appearance | | |
| | Example | | | | | | | | | | | |
| | G* | 105 | 106 | G* | 105 | 106 | G* | 105 | 106 | G* | 105 | 106 |
| HCl (37%) | 26.78 | 9.66 | 1.58 | little swelling | no | no | 72.19 | 26.14 | 3.39 | further | little | no |
| | | | | no | change | change | | | | swelling | swelling, | swelling, |
| | | | | discoloration | | | | | | no | slightly | slightly |
| | | | | | | | | | | discoloration | opaque | opaque |
| HNO₃ (10%) | 3.19 | 0.37 | 0.18 | no change | no | no | 10.88 | 0.91 | 0.45 | no change | no swelling, | no |
| | | | | | swelling, | swelling, | | | | | yellow | swelling, |
| | | | | | slightly | slightly | | | | | | yellow |
| | | | | | yellow | yellow | | | | | | |
| H₂SO₄ | 2.53 | 0.10 | 0.05 | no change | no | no | 7.44 | 0.39 | 0.12 | no change | no change | no |
| (30%) | | | | | change | change | | | | | | change |
| CH₃COOH | 1.96 | 0.43 | 0.26 | no swelling, | no | no | 5.33 | 1.03 | 0.42 | no swelling, | no swelling | no |
| (5%) | | | | but cloudy | swelling | swelling | | | | but cloudy | opaque | swelling |
| | | | | | slightly | slightly | | | | | | opaque |
| | | | | | opaque | opaque | | | | | | |
| Citric acid | 1.26 | 0.38 | 0.18 | no swelling, | no | no | 3.92 | 0.68 | 0.29 | no swelling, | no swelling | no |
| (10%) | | | | but cloudy | change | change | | | | but cloudy | slightly | swelling |
| | | | | | | | | | | | opaque | slightly |
| | | | | | | | | | | | | opaque |
| De- | 1.06 | 0.24 | 0.14 | no change | no | no | 2.78 | 0.63 | 0.34 | no swelling | no swelling | no |
| mineralized | | | | | change | change | | | | a bit cloudy | cloudy | swelling |
| Water | | | | | | | | | | | | cloudy |
| NaCl (30%) | 0.34 | 0.06 | 0.03 | no change | no | no | 1.02 | 0.30 | 0.09 | no change | no change | no |
| | | | | | change | change | | | | | | change |
| Soap | 1.84 | 0.34 | 0.19 | no change | no | no | 4.87 | 0.91 | 0.42 | no change | no swelling | no |
| solution | | | | | change | change | | | | | opaque | swelling |
| (1%) | | | | | | | | | | | | opaque |
| H₂O₂ (30%) | 5.95 | 0.81 | 0.35 | no change | no | no | 15.49 | 2.21 | 0.66 | no change | no swelling | no |
| | | | | | change | change | | | | | opaque | swelling |
| | | | | | | | | | | | | opaque |
| Na₂CO₃-Sol. | 0.93 | 0.26 | 0.11 | no change | no | no | 2.40 | 0.64 | 0.28 | no change | no swelling | no |
| (2%) | | | | | change | change | | | | | opaque | swelling |
| | | | | | | | | | | | | opaque |
| NaOH (10%) | 0.89 | 0.23 | 0.09 | no change | no | no | 1.89 | 0.44 | 0.17 | little cloudy | no swelling | no |
| | | | | | swelling | swelling | | | | on surface | opaque | swelling |
| | | | | | opaque | opaque | | | | | | opaque |
| NH₃ (28%) | 1.05 | 0.38 | 0.27 | no change | no | no | 2.06 | 0.92 | 0.69 | no swelling | no swelling, | no |
| | | | | | swelling, | swelling, | | | | yellowish, | opaque | swelling, |
| | | | | | slightly | opaque | | | | cloudy | | opaque |
| | | | | | opaque | | | | | | | |
| Mineral oil | 0.19 | 0.01 | 0.02 | no change | no | no | 0.46 | 0.01 | 0.02 | no change | no change | no |
| | | | | | change | change | | | | | | change |
| Diesel | 2.57 | 2.16 | 5.20 | no change | no | no | 6.99 | 5.59 | 14.07 | no change | no swelling | no |
| | | | | | change | change | | | | | slightly | swelling |
| | | | | | | | | | | | yellow | slightly |
| | | | | | | | | | | | | yellow |
| Toluol | 168.41 | 99.38 | 96.46 | strong swelling | strong | strong | 390.90 | 314.33 | 284.89 | further | further | further |
| | | | | no discoloration | swelling | swelling | | | | swelling, | swelling, | swelling, |
| | | | | | | | | | | tears easily | tears easily | tears |
| | | | | | | | | | | no | | easily |
| | | | | | | | | | | discoloration | | |
| DMF | 99.83 | 51.97 | 28.22 | strong swelling | swelling | little | 400.92 | 233.67 | 89.43 | further | further | strong |
| | | | | surface cracked | | swelling | | | | swelling, | swelling, | swelling |
| | | | | no discoloration | | | | | | tears easily | tears easily | |
| | | | | | | | | | | no | | |
| | | | | | | | | | | discoloration | | |
| Ethylacetate | 188.13 | 90.46 | 43.74 | strong swelling | strong | swelling | 447.28 | 294.33 | 154.22 | further | further | strong |
| | | | | surface cracked | swelling | | | | | swelling, | swelling, | swelling, |
| | | | | | | | | | | tears easily | tears easily | opaque |
| Acetone | 135.58 | 62.81 | 26.28 | strong swelling | swelling | little | 328.46 | 177.02 | 73.37 | further | strong | swelling |
| | | | | surface cracked | | swelling | | | | swelling, | swelling | |
| | | | | no discoloration | | | | | | tears easily | | |
| | | | | | | | | | | no | | |
| | | | | | | | | | | discoloration | | |
| Methanol | 46.69 | 8.71 | 2.40 | little swelling | no | no | 178.17 | 24.86 | 6.26 | further | little | no |
| | | | | no discoloration | change | change | | | | swelling no | swelling no | change |
| | | | | | | | | | | discoloration | discoloration | |
| Ethanol | 36.18 | 7.44 | 1.97 | little swelling | no | no | 124.87 | 21.22 | 5.62 | further | little | no |
| | | | | no discoloration | change | change | | | | swelling no | swelling no | change |
| | | | | | | | | | | discoloration | discoloration | |

As seen in Table 16, the two systems which incorporated NOBP into the formulations show less weight gain and better sample appearance after the exposure to most of the various medias than the comparative example which did not incorporate any NOBP.

For example: the elastomer based on pure NOBP chemistry (Example 106) showed the lowest weight increase when exposed to aqueous media (VE-water, NaCl solution, soap solution, and H₂O₂ solution), while the standard system based on pure polyether polyols exhibited the highest weight increase (Comparative Example I). The performance of the polyether/NOBP blended type fell in between (Example 105). Thus, it can be concluded that the pure NOBP containing systems and the NOBP/polyether polyol based soft elastomer systems show a significant improvement in chemical resistance against aqueous media compared to the standard polyether polyol based PU-gel system. The NOBP polyols have a more hydrophobic character compared to the polyether polyols, resulting in a more hydrophobic character of the gel systems made from NOBPs as well. The degree of chemical resistance is thereby directly correlated to the incorporated amount of NOBP polyol in the gel matrix.

For acidic and basic media, as observed with aqueous media, the systems made of pure NOBPs or polyether polyol/NOBP blends showed lower weight increases compared to the standard polyether polyol based system. Again this may be explained by the more hydrophobic character of the NOBP based polyol compared to polyether polyols. The more NOP polyol the PU-gel system contains the higher is the chemical resistance versus acidic or basic media.

All three of the systems tested showed significant swelling and weight gains in excess of 50% in toluene, DMF, ethylacetate, and acetone when immersed for a 24 hours period. The best chemical resistance was achieved by the pure NOBP based gel, followed by the polyether polyol/NOBP blended system. At a short exposure time of 3 hours the pure NOBP based system was the best candidate, with weight gains below 50% for DMF, weight gains in ethylacetate and acetone medium remained below 100%.

The immersion test at 24 hours in ethanol and methanol showed for the NOBP/polyether polyol blended system and for the pure NOBP based system weight gains below 50%, while the values for the standard polyether polyol based systems were higher than 100%. Also, the NOBP containing systems showed improvements in terms of chemical resistance compared to their polyether polyol based countertype.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

1. A polyurethane product comprising a reaction product of at least one first polyol composition and at least one prepolymer composition, wherein the prepolymer composition comprises the reaction product of at least a second polyol composition and at least one isocyanate composition, at least one of the first polyol composition and the second polyol composition comprises at least one polyol derived from a natural oil, and wherein the polyurethane product has a Shore 00 hardness as measured according to ASTM D 2240 of between about 0 Shore 00 and about 80 Shore 00 and is one of a gel and a soft eleastomer.

2. A method of forming a polyurethane product, the method comprising:
providing at least a first polyol composition;
forming at least one prepolymer composition by combing at least a second polyol composition with at least one isocyanate composition;
reacting the at least first polyol composition with the at least one prepolymer composition,
wherein at least one of the first polyol composition and the second polyol composition comprises at least one polyol derived from a natural oil and wherein the polyurethane product has a Shore 00 hardness as measured according to ASTM D 2240 of between about 0 Shore 00 and about 80 Shore 00 and is one of a gel and a soft eleastomer.

3. The polyurethane product of claim 1, wherein the at least one polyol derived from a natural oil comprises at least one of a hydroxymethylated fatty acid and a hydroxymethylated fatty acid ester.

4. The polyurethane product of claim 3, wherein the at least one polyol derived from a natural oil comprises the reaction product of at least one of a hydroxymethylated fatty acid and a hydroxymethylated fatty acid ester and an initiator compound having a OH functionality, primary amine functionality, secondary amine functionality, or combination OH, primary, or secondary amine functionality, of between about 2 and about 4.

5. The polyurethane product of claim 4, wherein the initiator compound is selected from ethylene glycol, 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, glycerine, trimethylol propane, 1,2,6-hexane triol, 1,2,4-butane triol, trimethylolethane, pentaerythritol, quinitol, mannitol, sorbitol, methyl glycoside, diethylene glycol, triethylene glycol, tetraethylene glycol, dipropylene glycol, dibutylene glycol and combinations thereof.

6. (canceled)

7. The polyurethane product of claim 1, wherein the at least one polyol derived from a natural oil comprises at least an aliphatic polyester polyol prepared by the condensation of at least one diol with adipic, glutaric, succinic, dimer acid, or combination thereof and

8. the at least one diol is selected from 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, and combinations thereof.

9. The polyurethane product of claim 1, wherein the at least one first polyol composition comprises the at least one polyol derived from a natural oil and

10. the at least one second polyol composition comprises the at least one polyol derived from a natural oil.

11. The polyurethane product of claim 7, wherein the at least one second polyol composition comprises at least one polyol derived from a natural oil, and which is the same as the at least one polyol derived from a natural oil of the first polyol composition.

12. The polyurethane product of claim 7, wherein the at least one second polyol composition comprises at least one polyol derived from a natural oil, and which is different from the at least one polyol derived from a natural oil of the first polyol composition.

13. The polyurethane product of claim 1, wherein at least one of the first polyol composition and the second polyol composition further comprises a polyether polyol having a nominal functionality of 1 to 8.

14. The polyurethane product of claim 1, wherein the polyurethane product has a Shore 00 hardness of less than about 50 Shore 00.

15. The polyurethane product of claim 1, further comprising at least one foil adhered to the at least one of a gel and a soft eleastomer.

16. The method of claim 2, wherein the at least one polyol derived from a natural oil comprises at least one of a hydroxymethylated fatty acid and a hydroxymethylated fatty acid ester.

17. The method of claim 3, wherein the at least one polyol derived from a natural oil comprises the reaction product of at least one of a hydroxymethylated fatty acid and a hydroxymethylated fatty acid ester and an initiator compound having a OH functionality, primary amine functionality, secondary amine functionality, or combination OH, primary, or secondary amine functionality, of between about 2 and about 4.

18. The method of claim 15, wherein the initiator compound is selected from ethylene glycol, 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, glycerine, trimethylol propane, 1,2,6-hexane triol, 1,2,4-butane triol, trimethylolethane, pentaerythritol, quinitol, mannitol, sorbitol, methyl glycoside, diethylene glycol, triethylene glycol, tetraethylene glycol, dipropylene glycol, dibutylene glycol and combinations thereof.

19. The method of claim 2, wherein the at least one polyol derived from a natural oil comprises at least an aliphatic polyester polyol prepared by the condensation of at least one diol with adipic, glutaric, succinic, dimer acid, or combination thereof and

20. the at least one diol is selected from 1,2- and 1,3-propylene glycol, 1,4- and 2,3-butane diol, 1,6-hexane diol, 1,8-octane diol, neopentyl glycol, cyclohexane dimethanol, 1,3-cyclohexane dimethanol and 1,4-cyclohexane dimethanol, 2-methyl-1,3-propane diol, and combinations thereof.

21. The method of claim 2, wherein the at least one first polyol composition comprises the at least one polyol derived from a natural oil and

22. the at least one second polyol composition comprises the at least one polyol derived from a natural oil.

23. The method of claim 17, wherein the at least one second polyol composition comprises at least one polyol derived from a natural oil, and which is the same as the at least one polyol derived from a natural oil of the first polyol composition.

24. The method of claim 17, wherein the at least one second polyol composition comprises at least one polyol derived from a natural oil, and which is different from the at least one polyol derived from a natural oil of the first polyol composition.

25. The method of claim 2, wherein at least one of the first polyol composition and the second polyol composition further comprises a polyether polyol having a nominal functionality of 1 to 8.

26. The method of claim 2, wherein the polyurethane product has a Shore 00 hardness of less than about 50 Shore 00.
